(19) United States
(12) Patent Application Publication
(10) Pub. No.: US 2007/0149485 A1
(43) Pub. Date: Jun. 28, 2007

(54) SELECTED DOSAGE FOR THE TREATMENT OF CARDIOVASCULAR AND RELATED PATHOLOGIES

(75) Inventor: Albert D. Friesen, Winnipeg (CA)

Correspondence Address: MERCHANT & GOULD PC, P.O. BOX 2903, MINNEAPOLIS, MN, 55402-0903, US

(73) Assignee: Medicure International, Inc., St. James (BB)

(21) Appl. No.: 11/564,192

(22) Filed: Nov. 28, 2006

Related U.S. Application Data
(60) Provisional application No. 60/803,298, filed on May 26, 2006.
(60) Provisional application No. 60/753,853, filed on Dec. 23, 2005.
(60) Provisional application No. 60/740,970, filed on Nov. 28, 2005.

(51) Int. Cl. A61K 31/675

(52) U.S. Cl. 514/89

(57) ABSTRACT

Method is provided of treating or preventing hypertrophy, hypertension, myocardial ischemia, ischemic heart disease, myocardial infarction, congestive heart failure, organ ischemia, tissue ischemia, acute coronary syndrome, unstable angina, ischemia reperfusion injury, preventing death subsequent to myocardial infarction, cerebral infarction, contractile dysfunction subsequent to myocardial infarction, or arrhythmia in a mammal with low doses of pyridoxine, pyridoxal-5′phosphate, pyridoxal or pyridoxamine. Compositions and kits for said method is also included.

### CROSS-REFERENCE TO RELATED APPLICATION

This application claims the benefit under 35 U.S.C. §119(e) of U.S. provisional application No. 60/740,970, filed on Nov. 28, 2005, U.S. provisional application No. 60/753,853, filed Dec. 23, 2005, and 60/803,298, filed May 26, 2006, the entire disclosures of which are hereby incorporated by reference.

### FIELD OF THE INVENTION

This invention relates to a method of treating or preventing hypertrophy, hypertension, congestive heart failure, myocardial ischemia, ischemic heart disease, myocardial infarction, acute coronary syndrome, unstable angina, ischemia in various organs and tissues, ischemia reperfusion injuries in various organs and tissues, and to treating cellular dysfunction, including arrhythmia and heart failure subsequent to myocardial infarction.

### BACKGROUND

Heart failure is the pathophysiological state in which the heart is unable to pump blood at a rate commensurate with the requirement of the metabolizing tissues or can do so only from an elevated filling pressure (increased load). When this condition leads to excess fluid accumulation, such as peripheral edema, it is referred to as congestive heart failure. Heart failure can occur subsequent to myocardial infarction, which is necrosis of a region of the myocardium caused by an interruption in the supply of blood to the heart, usually as a result of occlusion of a coronary artery.

When an excessive pressure or volume load is imposed on the ventricle, myocardial hypertrophy develops, providing a compensatory mechanism that permits the ventricle to sustain an increased load. However, a ventricle subjected to an abnormally elevated load for a prolonged period may fail to maintain compensation despite the presence of ventricular hypertrophy and pump failure may ultimately occur. When heart failure occurs in a ventricle, it is sometimes referred to as a ventricular failure.

Ischemia is caused when an organ or a part of the body fails to receive a sufficient blood supply. An organ that is deprived of a blood supply is said to be hypoxic. An organ will become hypoxic even when the blood supply temporarily ceases, such as during a surgical procedure or during temporary artery blockage. Ischemia leads to structural and functional abnormalities, such as arrhythmias, cell death, and ventricular remodeling. When the organ affected is the heart, this condition is known as ischemic heart disease, or myocardial ischemia.

Myocardial ischemia initially leads to abnormal electrical activity, which may generate an arrhythmia. When myocardial ischemia is of sufficient severity and duration, cell injury may progress to cell death i.e., myocardial infarction, and subsequently to heart failure, hypertrophy, or congestive heart failure. Other organs and tissues can be affected, such as the kidney, colon, pancreas, an arm, leg, etc. Ischemia of various organs and tissues is well known and characterized, and can cause structural and functional abnormalities including failure of the organ.

When blood flow resumes to an organ after temporary cessation, this is known as ischemic reperfusion of the organ. For example, reperfusion of an ischemic myocardium may counter the effects of coronary occlusion, a condition that leads to myocardial ischemia. Ischemic reperfusion to the myocardium may lead to reperfusion arrhythmia or reperfusion injury. The severity of reperfusion injury is affected by numerous factors, such as, for example, duration of ischemia, severity of ischemia, and speed of reperfusion. Conditions observed with ischemia reperfusion injury include neutrophil infiltration, necrosis, and apoptosis.

Lack of blood flow to the brain, through ischemia or any other heart-related disease, can cause stroke, which may lead to sensorimotor abnormalities such as paralysis, which can be permanent.

Ischemia and other heart diseases are often associated with angina, a heart condition marked by paroxysms of chest pain due to reduced oxygen to the heart.

Acute Coronary Syndrome is a term used to describe cardiovascular irregularities; the term encompasses a range of thrombolytic coronary artery diseases, including unstable angina, Q-wave myocardial infarction and non-Q-wave myocardial infarction.

Ischemic Heart Disease is also known as coronary artery disease. This type of disease is the result of narrowed coronary arteries which restricts the blood flow to the heart muscle thereby creating an ischemic environment.

Hypertension, or high blood pressure, is a condition where blood pressure is chronically elevated in a patient. Hypertension puts the heart and arteries under greater strain may eventually contribute to heart attack or stroke.

Coronary artery bypass grafting (CABG) effectively relieves angina, results in longer survival, and a better quality of life in specific subgroups of patients with obstructive coronary artery disease. Due to the high incidence of coronary artery disease worldwide, as well as the effectiveness of this surgical procedure, CABG surgery makes up one of the top ten most frequently performed procedures in North America and Europe. In the year 2000, the total volume of bypass surgery was over 280,000 in the 15 European Union countries according to the European Heart Survey and the National Registries of Cardiovascular Diseases and Patient Management. In the United States, it is estimated that over 700,000 CABG procedures are performed per year.

Despite the benefits of CABG surgery, patients undergoing these procedures may also suffer serious adverse outcomes including operative mortality, myocardial infarction, acute coronary syndrome, unstable angina, ventricular failure, life-threatening arrhythmia, renal insufficiency, cognitive decline, and stroke. Some of the proposed causes of cardiovascular morbidity and mortality after CABG include perioperative ischemia, inadequate myocardial protection, and reperfusion injury. The impact of these serious complications is significant. Incidence rates of death and MI following CABG surgery range from 5% to 12% depending on risk status. Results from large clinical trials have recently demonstrated the importance of neurologic deficits (known as cognitive decline) as a problematic outcome of CABG. These deficits include impairment of memory, psychomotor, visuospatial, attention, and language abilities as measured by neuropsychological testing as well as the sensorimotor abnormalities associated with stroke.

It is difficult to assess whether myocardial infarction has, in fact, occurred. The “gold standard” for measuring myocardial infarction is a biochemical assay which looks at creatine kinase-MB (CK-MB) levels in the blood. Elevated CK-MB levels are correlated with myocardial infarction. For example, a CK-MB level of 10× ULN (50 ng/ml) or above is often used as an indicator of myocardial necrosis. Other experts, and other studies, look at varying “cut-offs” of CK-MB elevation as indicative of the existence of myocardial necrosis, with 50, 70, and 100 ng/ml cut-offs appearing most frequently in the literature.

Cerebral ischemic injury and cognitive impairment are serious and frequently occurring complications of coronary artery bypass graft (CABG) surgery (van Dijk et al, J. Thorac. Cardiovasc. Surg. 2000 Oct.; 120 (4): 632-9). Approximately two thirds of cardiac surgery patients experience deterioration in performance on neurocognitive tests in the early postoperative period, and these deficits can last for months or years (Taggart et al, Curr. Opin. Cardiol. 2001 Sep.; 16(5):271-6). Pyridoxal-5′-phosphate has been shown to have neuroprotective effects in an animal model of ischemic brain injury (Wang, et al, J. Neurosurg. 2005 Jul.; 103(1):165-9).

Pyridoxal-5′-phosphate (3-hydroxy-2-methyl-5-[(phosphonooxy)methyl]-4-pyridine-carboxaldehyde, or “P5P”) is a naturally occurring metabolite of pyridoxine (vitamin B6) and is formed in mammalian cells by phosphorylation and oxidation reactions. A recent evaluation demonstrated that P5P inhibits adenosine triphosphate (ATP) induced calcium ion influx into cells. Results suggest that this action is due to the antagonism of purinergic receptors known as P2 purinoceptors. It is hypothesized that P5P might prevent or reduce tissue damage during ischemia and reperfusion episodes by blocking calcium influx.

P5P can be chemically synthesized in a number of ways, for example, by the action of ATP on pyridoxal, by the action of phosphorus oxychloride on pyridoxal in aqueous solution, and by phosphorylation of pyridoxamine with concentrated phosphoric acid followed by oxidation.

P5P and other related compounds such as pyridoxine, pyridoxal and pyridoxamine, have been previously shown to be effective in the treatment of hypertrophy, congestive heart failure, myocardial ischemia, ischemia reperfusion injuries in an organ, and arrhythmia and contractile dysfunction subsequent to myocardial infarction. P5P has also been used therapeutically as an enzyme cofactor vitamin.

P5P is described in U.S. Pat. No. 6,043,259 herein incorporated by reference, as having an effective enteral or parenteral dosage range of about 0.5 to about 100 mg/kg of patient body weight, for treating hypertrophy, hypertension, congestive heart failure, myocardial ischemia, ischemia reperfusion injury and cellular dysfunction.

### SUMMARY OF THE INVENTION

The present invention includes methods and compositions for treating cardiovascular diseases and diseases related thereto. In one aspect, the invention includes a method for treating hypertrophy, hypertension myocardial ischemia, ischemic heart disease, organ ischemia, such as kidney ischemia, tissue ischemia, acute coronary syndrome, unstable angina, myocardial infarction, cerebral infarction, congestive heart failure, cognitive decline (such as cognitive decline following ischemia or following CABG surgery), ischemia reperfusion injury and cellular dysfunction (including arrhythmia, heart failure subsequent to myocardial infarction, cell death, and ventricular remodeling) in mammals that includes orally administering to the mammal a therapeutic amount in a range of about 1-10 mg/kg of the mammal's body weight per day of a compound comprising pyridoxine, pyridoxal-5′-phosphate, pyridoxal, or pyridoxamine. In one aspect, the compound is pyridoxal-5′-phosphate. In another aspect, administration can treat or prevent contractile dysfunction subsequent to myocardial infarction. In another aspect, administration can treat or prevent cerebral infarction (stroke). The cerebral infarction (stroke) can be a cerebral infarction (stroke) occurring subsequent to myocardial infarction.

In another aspect, the invention includes a method of preventing death subsequent to myocardial infarction in a mammal comprising: orally administering to said mammal a therapeutic amount in a range of about 1-10 mg/kg of the mammal's body weight per day of a compound comprising pyridoxine, pyridoxal-5′-phosphate, pyridoxal or pyridoxamine. In one embodiment, the compound is pyridoxal-5′-phosphate.

In another aspect, the compound can be administered prior to, or following, a procedure such as a bypass surgery, thrombolysis, endarterectomy, CABG and angioplasty. Another aspect of the invention includes the methods as described above wherein the mammal is a human and said oral therapeutic amount is in a range of about 100 mg per day to about 1000 mg per day, about 200 mg per day to about 300 mg per day, about 250 mg per day, or about 750 mg per day.

In another aspect, the invention includes a method for treating hypertrophy, myocardial ischemia, ischemic heart disease myocardial infarction, congestive heart failure, organ ischemia such as kidney ischemia, tissue ischemia, acute coronary syndrome, unstable angina, ischemia reperfusion injury, cognitive decline (such as cognitive decline following ischemia or CABG surgery), and cellular dysfunction (including arrhythmia, heart failure subsequent to myocardial infarction, cell death, and ventricular remodeling) in mammals that includes parenterally administering to the mammal a therapeutic amount in a range of about 0.028 to about 0.57 mg/kg of the mammal's body weight per day of a compound selected from pyridoxine, pyridoxal-5′-phosphate, pyridoxal, and pyridoxamine. In one aspect, the compound is pyridoxal-5′-phosphate. In another aspect, administration can treat or prevent contractile dysfunction subsequent to myocardial infarction. In another aspect, administration can treat or prevent cerebral infarction (stroke). The cerebral infarction (stroke) can be a cerebral infarction (stroke) occurring subsequent to myocardial infarction.

In another aspect, the invention includes a method of preventing death subsequent to myocardial infarction in a mammal comprising: parenterally administering to said mammal a therapeutic amount in a range of about 0.028 to about 0.57 mg/kg of the mammal's body weight per day of a compound selected from pyridoxine, pyridoxal-5′-phosphate, pyridoxal, or pyridoxamine. In one embodiment, the compound is pyridoxal-5′-phosphate.

In another aspect, the compound can be administered prior to, or following, a procedure such as bypass surgery, thrombolysis, CABG, angioplasty, or endarterectomy, such as a carotid endarterectomy.

Another aspect of the invention includes the methods as described above wherein the mammal is a human and said parenteral therapeutic amount is in a range of about 2.00 mg per day to about 40.00 mg per day, about 5.00 mg per day, or about 23.33 mg per day.

Parenteral administration may be an intravenous injection. Intravenous injection may be either a bolus or a continuous injection. Oral administration may be an enteral administration, such as a liquid, pill or capsule to be swallowed.

Another aspect of the present invention includes a method as herein described further comprising administration of one or more additional therapeutic compounds.

One aspect includes treating or preventing hypertrophy by administering P5P combined with an additional administration of one or more additional compounds comprising a calcium channel blocker, a vasodilator, a diuretic, an alpha-blocker, or a beta-blocker.

Another aspect includes treating or preventing hypertension by administering P5P combined with an additional administration of one or more additional compounds comprising an ACE inhibitor, an angiotensin II receptor antagonist, a renin inhibitor, an endothelin selective receptor antagonist, a diuretic, a alpha blocker, a calcium channel blocker, and a vasodilating agent. When treating or preventing hypertension, a method may include administering P5P combined with a drug commonly used for closely related diseases, such as diabetes and hyperlipidemia.

Another aspect includes treating or preventing myocardial ischemia by administering P5P combined with an additional administration of one or more additional compounds comprising a HMG-CoA Reductase inhibitor, a vasodilating agent, a diuretic, an ACE inhibitor, a beta-blocker, an angiotensin II receptor antagonist, a calcium channel blocker, an anticoagulant, an adenosine diphosphate receptor antagonist, a glycoprotein IIb/IIIa receptor antagonist, or an alpha blocker.

Another aspect includes treating or preventing ischemic heart disease and the administration is combined with an additional administration of one or more additional compounds selected from the group consisting of a HMG-CoA Reductase inhibitor, a vasodilating agent, a diuretic, an ACE inhibitor, a beta-blocker, an angiotensin II receptor antagonist, a calcium channel blocker, an anticoagulant, an adenosine diphosphate receptor antagonist, a glycoprotein IIb/IIIa receptor antagonist and an alpha blocker.

Another aspect includes treating or preventing congestive heart failure by administering P5P combined with an additional administration of one or more additional compounds comprising a cardiotonic agent, a cardiac glycoside, a vasodilating agent, a nitrate, a nitrite, an ACE inhibitor, an angiotensin II receptor antagonist, a mineralocorticoid, a diuretic, or an alpha-adrenergic receptor antagonist.

Another aspect treating or preventing acute coronary syndrome by administering P5P combined with an additional administration of one or more additional compounds comprising a GB IIb/IIIa receptor antagonists, an anti-platelet agent, a calcium channel blocker, a beta blocker, a nitrate, or an anticoagulant.

Another aspect includes treating or preventing unstable angina by administering P5P combined with an additional administration of one or more additional compounds comprising a HMG-CoA Reductase Inhibitor, a GP IIb/IIIa Receptor antagonist, an anticoagulant, an adenosine diphosphate receptor antagonist, or heparin.

Another aspect includes treating or preventing ischemia reperfusion injury by administering P5P combined with an additional administration of one or more additional compounds comprising a HMG-CoA Reductase inhibitor, a vasodilating agent, a diuretic, an ACE inhibitor, a beta-blocker, an angiotensin II receptor antagonist, a calcium channel blocker, an anticoagulant, an adenosine diphosphate receptor antagonist, a glycoprotein IIb/IIIa receptor antagonist, an alpha blocker, or an anticoagulant.

Another aspect includes treating or preventing cerebral infarction (stroke) by administering P5P combined with an additional administration of one or more additional compounds comprising a HMG-CoA reductase inhibitor, an ACE inhibitor, an angiotensin II receptor antagonist, an adenosine diphosphate receptor antagonist, a glycoprotein IIb/IIIa receptor antagonist, an anticoagulant, agent, or a calcium channel blocking agent.

Another aspect includes treating or preventing arrhythmia by administering P5P combined with an additional administration of one or more additional compounds comprising an antiarrhythmic, heparin, warfarin, and digoxin.

Another aspect includes treating or preventing myocardial infarction by administering P5P combined with an additional administration of one or more additional compounds comprising a HMG-CoA reductase inhibitor, an ACE inhibitor, an anticoagulant, an adenosine diphosphate receptor antagonist, a glycoprotein IIb/IIIa receptor antagonist, an alpha-adrenergic receptor antagonist, a beta-blocker, an antiarrhythmic, a hypotensive agent, a direct vasodilator, or a calcium channel blocking agent.

Another aspect of the present invention includes a composition formulated for oral administration comprising about 100 to about 1000 mg of pyridoxal-5′-phosphate, about 250 mg of pyridoxal-5′-phosphate, about 750 mg of pyridoxal-5′-phosphate, or about 100 to about 1000 mg, about 250 mg, or about 750 mg of any of the other compounds discussed above, optionally further comprising a pharmaceutically acceptable excipient, and optionally further comprising an additional therapeutic compound as described above.

Another aspect of the present invention includes a composition formulated for parenteral administration comprising about 2.00 mg to 40.00 mg of pyridoxal-5′-phosphate, about 5.00 mg of pyridoxal-5′-phosphate, about 23.33 mg of pyridoxal-5′-phosphate, or about 2.00 mg to 40.00 mg, about 5.00 mg, or about 23.33 mg of any of the other compounds discussed above, optionally further comprising a pharmaceutically acceptable excipient.

In another aspect, a pharmaceutical composition includes a pharmaceutically acceptable carrier and a therapeutically effective amount, from about 200 to about 1000 mg (oral) or about 2.00 mg to about 40.00 mg (parenteral), of a compound comprising pyridoxal-5′-phosphate, pyridoxine, pyridoxal, or pyridoxamine for treating hypertrophy, hypertension, congestive heart failure, myocardial ischemia, ischemic heart disease, organ ischemia such as kidney ischemia, tissue ischemia, acute coronary syndrome, unstable angina, ischemia reperfusion injury and cellular dysfunction or for preventing post-operative complications after surgical procedures (such as open heart surgery, for example, CABG), such post-operative complications including hypertrophy, congestive heart failure, myocardial ischemia, organ ischemia such as kidney ischemia, tissue ischemia, acute coronary syndrome, unstable angina, ischemia reperfusion injury, cognitive decline and cellular dysfunction (including arrhythmia, heart failure subsequent to myocardial infarction, cell death, and ventricular remodeling). In one aspect, the compound is pyridoxal-5′-phosphate.

In another aspect, a pharmaceutical composition includes a pharmaceutically acceptable carrier and a therapeutically effective amount, from about 200 to about 300 mg (oral) or about 3.00 mg to about 6.00 mg (parenteral), of a compound comprising pyridoxal-5′-phosphate, pyridoxine, pyridoxal, or pyridoxamine for treating hypertrophy, congestive heart failure, myocardial ischemia, organ ischemia such as kidney ischemia, tissue ischemia, acute coronary syndrome, unstable angina, ischemia reperfusion injury and cellular dysfunction or for preventing post-operative complications after surgical procedures (such as open heart surgery, for example, CABG), such post-operative complications including hypertrophy, congestive heart failure, myocardial ischemia, organ ischemia such as kidney ischemia, tissue ischemia, acute coronary syndrome, unstable angina, ischemia reperfusion injury cognitive decline and cellular dysfunction (including arrhythmia, heart failure subsequent to myocardial infarction, cell death, and ventricular remodeling). In one aspect, the compound is pyridoxal-5′-phosphate.

In another aspect, a pharmaceutical composition includes a pharmaceutically acceptable carrier and a therapeutically effective amount, of about 250 mg (oral) or about 5.00 mg (parenteral), of a compound comprising pyridoxal-5′-phosphate, pyridoxine, pyridoxal, or pyridoxamine for treating hypertrophy, hypertension, congestive heart failure, myocardial ischemia, ischemic heart disease, organ ischemia such as kidney ischemia, tissue ischemia, acute coronary syndrome, unstable angina, ischemia reperfusion injury and cellular dysfunction, or for preventing post-operative complications after surgical procedures (such as open heart surgery, for example, CABG), such post-operative complications including hypertrophy, congestive heart failure, myocardial ischemia, organ ischemia such as kidney ischemia, tissue ischemia, acute coronary syndrome, unstable angina, ischemia reperfusion injury, cognitive decline and cellular dysfunction (including arrhythmia, heart failure subsequent to myocardial infarction, cell death, and ventricular remodeling). In one aspect, the compound is pyridoxal-5′-phosphate.

Another aspect includes a kit comprising (a) a pharmaceutical preparation for oral administration comprising a compound comprising pyridoxine, pyridoxal, pyridoxal-5′-phosphate, or pyridoxamine; and (b) instructions for the administration of said preparation, said instructions specifying that said preparation should be administered in a dosage range of about 100 mg to about 1000 mg per day. In one aspect, the compound is pyridoxal-5′-phosphate. In a further aspect, the instructions may further specify that the preparation should be administered prior to or during a procedure selected from the group consisting of bypass surgery, thrombolysis, endarterectomy, CABG, and angioplasty. In yet a further aspect, the instructions may further specify that the preparation should be administered after a procedure selected from the group consisting of bypass surgery, thrombolysis, endarterectomy, CABG, and angioplasty. In a further aspect, the instructions may further specify that said preparation should be administered in a dosage range of about 200 mg to about 300 mg per day. In another aspect, the instructions may further specify that said preparation should be administered in a dosage of about 250 mg per day. In another aspect, the instructions may further specify that said preparation should be administered in a dosage of about 750 mg per day. In another aspect, the instructions may further specify that the preparation should be administered in combination with another therapeutic compound as described herein.

Yet another aspect of the present invention includes a kit comprising (a) a pharmaceutical preparation for parenteral administration comprising a compound selected from pyridoxine, pyridoxal, pyridoxal-5′-phosphate, and pyridoxamine; and (b) instructions for administering said preparation; said instructions specifying that said preparation should be administered in a dosage range of about 2.00 mg to about 40.00 mg per day. In a further aspect, the compound is pyridoxal-5′-phosphate. In a further aspect, the instructions may further specify that the preparation should be administered prior to or during a procedure selected from the group consisting of bypass surgery, thrombolysis, endarterectomy, CABG, and angioplasty. In another aspect, the instructions may further specify that the preparation should be administered after a procedure selected from the group consisting of bypass surgery, thrombolysis, endarterectomy, CABG, and angioplasty. In another aspect, the instructions may further specify that said preparation should be administered in a dosage range of about 3.00 mg to about 6.00 mg per day. In another aspect, the instructions may further specify that said preparation should be administered in a dosage of about 5.00 mg per day. In another aspect, the instructions may further specify that said preparation should be administered in a dosage of about 23.33 mg per day. In another aspect, the instructions may further specify that the preparation should be administered in combination with another therapeutic compound, as described herein. Another aspect includes a kit comprising both an oral and a parenteral dosage form, combined with instructions further specifying that the parenteral dosage form should be administered, followed by administration of the oral form.

### DETAILED DESCRIPTION

Although P5P is effective throughout the dose range disclosed in the prior art, surprisingly and paradoxically, P5P is more effective at the lower end of this dosage range, as compared to the higher end of the dosage range, especially when clinically accepted indicators of myocardial ischemia are used. Surprisingly an oral administration of a dosage range of 200-300 mg per day, for example, 250 mg per day, in humans, is more effective at prevention of post-CABG cardiovascular morbidity and mortality than higher dosages. This finding defies expectations of the current state of the art and the biochemical methods of action of P5P currently hypothesized. Oral dosages of 250 mg per day and 750 mg per day are effective at prevention of post-CABG cardiovascular morbidity and mortality in humans, and therefore hypothesize that a dosage range of 200 mg per day to 1000 mg per day, administered orally, or a pharmacokinetically equivalent dosage administered parenterally, for example, an IV dosage providing a mean AUC₍₀₋₂₄ₕ₎ of about 2 to about 15 ug.h/ml should be effective in reducing post-operative complications, morbidity and mortality, and should be appropriate post-operative treatment protocol for any form of invasive heart surgery, such as CABG. The IV dosage may 2.60±0.87 μg.h/ml, or 12.8±15.4 μg.h/ml.

For an average 70 kg person, a total IV dose of 5.00 mg is roughly equivalent to a 250 mg pill-form enteral administration, a total IV dose of 23.33 mg is roughly equivalent to a 750 mg pill-form enteral administration, and an IV dose range of 2.00-40.00 mg is roughly equivalent to a pill-form enteral administration range of 100-1000 mg.

Relatively low concentrations of P5P can be used advantageously in the treatment of the above-identified diseases and conditions. “Treatment” and “treating” as used herein include preventing, inhibiting, and alleviating cardiovascular diseases and related symptoms as well as healing the ischemia-related conditions or symptoms thereof affecting mammalian organs and tissues. Thus a composition of the present invention can be administered in a therapeutically effective amount to a patient before, during, and after any above-mentioned condition arises. As an example, for instance, a composition of the present invention can be administered prior to ischemia to prevent, inhibit, or protect against ischemia reperfusion injuries and cellular dysfunction of organs and tissues, such as kidney ischemia. Alternatively, a composition of the invention can be administered during or following ischemia (including during or following reperfusion) to alleviate or heal ischemia reperfusion injuries and cellular dysfunction of organs and tissues. In one embodiment, a composition of the invention can be administered after invasive surgery which is known to trigger these injuries, such as a CABG treatment. P5P related compounds, such as pyridoxal, pyridoxamine and pyridoxine, can likely be used in similarly comparatively low dosages as compared to the expected and previously taught dosages for those compounds. Dosages for these P5P related compounds can be readily determined, applying the present teachings to the known dosage and safety/effectiveness of these compounds, if necessary, combined with routine experimentation.

An embodiment of the invention includes a method of treating, preventing, or protecting against hypertrophy, hypertension, congestive heart failure, myocardial ischemia, ischemic heart disease, ischemia reperfusion injury, organ ischemia such as kidney ischemia, tissue ischemia, acute coronary syndrome, unstable angina, ischemia-related injuries, and cellular dysfunction in mammals comprising administering to the mammal a therapeutic amount of a compound comprising pyridoxal-5′-phosphate, pyridoxine, pyridoxal, or pyridoxamine. Cellular dysfunction may include an arrhythmia of the heart or heart failure resulting from myocardial infarction. A “therapeutic amount” as used herein includes a prophylactic amount, for example, an amount effective for preventing or protecting against ischemia-related conditions, and amounts effective for alleviating or healing ischemia-related conditions.

A therapeutic amount of a compound comprising pyridoxal-5′-phosphate, pyridoxine, pyridoxal, or pyridoxamine for treating hypertrophy, hypertension, congestive heart failure, myocardial ischemia, ischemic heart disease, organ ischemia such as kidney ischemia, tissue ischemia, acute coronary syndrome, unstable angina, ischemia reperfusion injury and cellular dysfunction preferably includes ae range of about 1-10 mg/kg of a patient's body weight, such as in the range of about 100-1000 mg per daily dose, a range of about 200-300 mg per daily dose, about 250 mg per daily dose, or optionally, about 750 mg per daily dose.

Parenterally administering a therapeutic amount of a compound comprising pyridoxal-5′-phosphate, pyridoxine, pyridoxal, or pyridoxamine for treating hypertrophy, congestive heart failure, myocardial ischemia, ischemic heart disease, organ ischemia such as kidney ischemia, tissue ischemia, acute coronary syndrome, unstable angina, cognitive decline, ischemia reperfusion injury and cellular dysfunction preferably is in the range of about 0.028 to 0.57 mg/kg of a patient's body weight, such as in the range of about 2.00 to about 40.00 mg per daily dose, the range of about 3.00-6.00 mg per daily dose, or about 5.00 mg per daily dose, or optionally, about 23.33 mg per daily dose.

A compound comprising pyridoxal-5′-phosphate, pyridoxine, pyridoxal, or pyridoxamine may be administered for periods of short and long durations depending on the condition treated.

A therapeutic amount of a compound comprising pyridoxal-5′-phosphate, pyridoxine, pyridoxal, or pyridoxamine for particularly treating ischemia-related conditions can be administered before, during, or following ischemia (including during or following reperfusion), as well as continually for some period spanning from pre- to post-ischemia. For example, a compound may be administered prior to procedures, including bypass surgery, thrombolysis, endarterectomy, and angioplasty, and prior to any other procedures that require interruption and then resumption of blood flow to any organ. A compound comprising pyridoxal-5′-phosphate, pyridoxine, pyridoxal, or pyridoxamine can also be taken during or after said heart procedure. Additionally, a compound comprising pyridoxal-5′-phosphate, pyridoxine, pyridoxal, or pyridoxamine may be taken on a regular basis to protect against cellular dysfunction arising from arrhythmia and heart failure.

A compound comprising pyridoxal-5′-phosphate, pyridoxine, pyridoxal, or pyridoxamine can be administered on its own, or in combination with a known therapeutic. When administered in combination, a combination may be one combined delivery, for example, an orally administered tablet containing both the compound of the present invention and a known therapeutic, or a combination may be two separate administrations, either administered at the same time or at different times. For example, when treating or preventing hypertrophy, a compound comprising pyridoxal-5′-phosphate, pyridoxine, pyridoxal, or pyridoxamine can be taken in combination with one or more of a calcium channel blocker, vasodilator, diuretic, alpha-blocker, or beta-blocker.

When treating or preventing myocardial ischemia, a compound comprising pyridoxal-5′-phosphate, pyridoxine, pyridoxal, or pyridoxamine can be taken in combination with one or more anticoagulant, such as enoxaparin sodium, bivalirudin, argatroban, or heparin, dalteparin sodium, HMG-CoA Reductase inhibitors, such as atorvastatin, vasodilating agents, such as dipyridamole, glycoprotein IIb/IIIa receptor antagonists such as abciximab, eptifibate, or tirofiban hydrochloride, or adenosine diphosphate receptor antagonists such as ticlopidine or clopidogrel bisulfate.

When treating or preventing congestive heart failure, a compound comprising pyridoxal-5′-phosphate, pyridoxine, pyridoxal, or pyridoxamine can be taken in combination with one or more cardiotonic agents, cardiac glycosides such as digoxin, inamrinone, lactate, milrinone lactate, vasodilating agents, nitrates and nitrites such as isosorbide dinitrate or isosorbide mononitrate, nesiritide, ACE inhibitors such as captopril, enalaprilat/enalapril maleate, benazepril hydrochloride, perindopril erbumine, trandolapril, lisinopril, moexipril hydrochloride, quinapril hydrochloride, or ramipril, angiotensin II receptor antagonists, such as candesartan cilexetil, eprosartan mesylate, irbesartan, losartan potassium, olmesartan medoxomil, telmisartan, or valsartan, mineralocorticoids, such as eplerenone or spironolactone, diuretics, such as amiloride hydrochloride or spironolactone, or alpha-adrenergic receptor antagonists.

When treating or preventing acute coronary syndrome, a compound comprising pyridoxal-5′-phosphate, pyridoxine, pyridoxal, or pyridoxamine of the present invention can be taken in combination with one or more GP IIb/IIIa receptor antagonists, such as tirofiban hydrochloride.

When treating or preventing unstable angina, a compound comprising pyridoxal-5′-phosphate, pyridoxine, pyridoxal, or pyridoxamine can be taken in combination with one or more HMG-CoA Reductase inhibitor, such as atorvastatin calcium or lovastatin, GP IIb/IIIa Receptor antagonists such as tirofiban hydrochloride with heparin and aspirin, isosorbide mononitrate, anticoagulants such as enoxaparin sodium, adenosine diphosphate receptor antagonists such as heparin or ticlopidine hydrochloride.

When treating or preventing ischemia reperfusion injury, a compound comprising pyridoxal-5′-phosphate, pyridoxine, pyridoxal, or pyridoxamine can be taken in combination with one or more ACE inhibitors, angiotensin II receptor antagonists, adenosine diphosphate receptor antagonists, such as ticlopidine or clopidogrel bisulfate, glycoprotein IIb/IIIa receptor antagonists, such as abciximab, eptifibate, or tirofiban hydrochloride, anticoagulants, such as bivalirudin, argatroban or heparin, calcium channel blocker, beta-adrenergic receptor antagonist, vasodilator, diuretic, alpha-adrenergic receptor antagonist, or antioxidants.

When treating or preventing cerebral infarction (stroke), a compound comprising pyridoxal-5′-phosphate, pyridoxine, pyridoxal, or pyridoxamine can be taken in combination with HMG-CoA Reductase Inhibitors such as pravastatin sodium, simvastatin, ACE inhibitors such as ramipril, Angiotensin II receptor antagonists such as losartan potassium, adenosine diphosphate receptor antagonists such as ticlopidine hydrochloride or clopidogrel bisulfate, aspririn, anti-thrombotic agents, calcium channel blocking agents, dihydropyridines such as nimodipine, beta-adrenergic receptor antagonists.

When treating or preventing arrhythmia, a compound comprising pyridoxal-5′-phosphate, pyridoxine, pyridoxal, or pyridoxamine may be used in combination with antiarrhythmics, such as Class Ia antiarrhythmics (for example disopyramide phosphate, procainamide hydrochloride, quinidine gluconate, quinidine sulfate), Class Ib antiarrhythmics (for example, lidocaine hydrochloride, mexiletine hydrochloride), Class Ic antiarrhythmics (for example encainide hydrochloride, flecainide acetate, propafenone hydrochloride), Class III antiarrhythmics (for example, amiodarone hydrochloride or sotalol), Class II antiarrhythmics (such as propanolol or esmolol acebutolol), Class IV antiarrhythmics (such as verapamil hydrochloride or diltiazem), heparin, warfarin, or digoxin.

When treating or preventing myocardial infarction, a compound comprising pyridoxal-5′-phosphate, pyridoxine, pyridoxal, or pyridoxamine may be used in combination with HMG-CoA Reductase inhibitors, such as atorvastatin calcium, lovastatin, pravastatin sodium, simvastatin, ACE iihibitors, such as lisinopril or ramapril, anticoagulants such as bivalirudin, argatroban, heparin, or warfarin, adenosine diphosphate receptor antagonists, such as clopidogrel bisulfate or ticlopidine, aspirin, GP IIb/IIIa receptor antagonists, such as tirofiban hydrochloride, eptifibate, abciximab, or tirofiban hydrochloride with heparin and aspirin (for non Q-wave myocardial infarction), anti-oxidants, alpha-adrenergic receptor antagonists, beta-blockers, antiarrythmics, such as class II antiarrhythmics like sotalol, hypotensive agents, direct vasodilators such as sodium nitroprusside, calcium channel blocking agents, dihydropyridines such as nifedipine, calcium channel blocking agents, such as diltiazem hydrochloride, verapamil hydrochloride, or anticoagulants such as enoxaparin sodium, heparin, argatroban, or bivalirudin.

By way of illustration, the following describes administration to a human of a pharmaceutical composition comprising P5P for treating ischemia. When a human is presented for a procedure, for example, bypass surgery, CABG, thrombolysis, endarterectomy, or angioplasty, or for a procedure requiring interruption of blood flow to any organ, an aqueous solution comprising P5P in a therapeutic amount can be given intravenously, immediately prior to surgery and then throughout a patient's hospitalization. Alternatively, a phannaceutical composition comprising P5P can be given immediately prior to surgery and then continuously for up to 30 days following surgery.

Similarly, a human may be administered an oral or parenteral dose of P5P beginning with onset of symptoms of ischemia-related conditions through a surgical procedure. Furthermore, a human at risk for arrhythmia or heart failure may be administered a regular oral or parenteral dose of P5P to protect against cellular dysfunction.

A pharmaceutical composition of the present invention includes a composition suitable for treating hypertrophy, hypertension, congestive heart failure, myocardial ischemia, ischemic heart disease, organ ischemia such as kidney ischemia, tissue ischemia, acute coronary syndrome, unstable angina, ischemia reperfusion injury, cognitive decline and cellular dysfunction. A phanmaceutical composition comprises a pharmaceutically acceptable carrier and a compound comprising pyridoxal-5′-phosphate, pyridoxine, pyridoxal and pyridoxamine. A phannaceutically acceptable carrier includes, but is not limited to, physiological saline, ringers, phosphate buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. A selected compound may be P5P. Advantageous pharmaceutical compositions include those that are made specifically for the oral treatment dosage levels discovered herein, for example, a pharmaceutical composition for oral administration comprising about 250 mg of P5P or an other compound of the invention, a phannaceutical composition for oral administration comprising about 750 mg of P5P or another compound of the invention, or a pharmaceutical composition for oral administration comprising about 100 to about 1000 mg of P5P or another compound of the invention.

Other advantageous pharmaceutical compositions include those that are made specifically for the parenteral treatment dosage levels discovered herein, for example, a pharmaceutical composition for parenteral administration comprising about 5.00 mg of a compound comprising pyridoxal-5′-phosphate, pyridoxine, pyridoxal, or pyridoxamine, a pharmaceutical composition for parenteral administration comprising about 23.33 mg of a compound comprising pyridoxal-5′-phosphate, pyridoxine, pyridoxal, or pyridoxamine, or a phannaceutical composition for parenteral administration comprising about 2.00 to about 40.00 mg of a compound comprising pyridoxal-5′-phosphate, pyridoxine, pyridoxal, or pyridoxamine.

Phannaceutical compositions may be administered orally or parenterally. Parenteral administration includes subcutaneous, intramuscular, intradermal, intramammary, intravenous, and other administrative methods known in the art. Parenteral administration may be bolus or continuous infusion. Oral administration includes enteral administration of solution, tablets, sustained release capsules, enteric coated capsules, and syrups. When administered, a pharmaceutical composition should be at or near body temperature.

Methods of preparing pharmaceutical compositions containing a phannaceutically acceptable carrier and a therapeutic compound comprising P5P, pyridoxine, pyridoxal and pyridoxamine are well known. As an illustration, a method of preparing a pharmaceutical composition containing P5P will be described.

An embodiment of the invention also includes pharmaceutically acceptable salts of a compound comprising pyridoxal-5′-phosphate, pyridoxine, pyridoxal, or pyridoxamine. A compound comprising pyridoxal-5′-phosphate, pyridoxine, pyridoxal, or pyridoxamine is capable of forming both pharmaceutically acceptable acid addition and/or base salts. Pharmaceutically acceptable acid addition salts include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and di-carboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glucamine, etc. (see Berge et al., J Pharmaceutical Science, 66: 1-19 (1977). The term “pharmaceutically acceptable salts” also includes any pharmaceutically acceptable base salt including, but not limited to, amine salts, trialkyl amine salts and the like. Such salts can be formed quite readily by those skilled in the art using standard techniques.

An acid addition salt can prepared by contacting a free base form with a sufficient amount of a desired acid to produce a salt in the conventional manner. A free base form may be regenerated by contacting a salt form with a base and isolating a free base in a conventional manner. A free base form differs from their respective salt forms somewhat in certain physical properties such as solubility in polar solvents, but otherwise salts are equivalent to their respective free base for purposes of the present invention. Base salts are formed with metals or amines, such as alkali and alkaline earth metals or organic amines. Examples of metals used as cations include, but are not limited to, sodium, potassium, magnesium, and calcium. Examples of suitable amines are N,N′-dibenzylethylenediamine, chloroprocaine, choline, diethanolamine, ethylenediamine, N-methylglucamine, and procaine.

Some of the compounds described herein contain one or more asymmetric centers and may thus give rise to enantiomers, diastereomers, and other stereoisomeric forms which may be defined in terms of absolute stereochemistry as (R)- or (S)-. The present invention is meant to include all such possible diastereomers and enantiomers as well as their racemic and optically pure forms. Optically active (R)- and (S)-isomers may be prepared using chiral synthons or chiral reagents, or resolved using conventional techniques. When a compound described herein contains a center of geometric asymmetry, and unless specified otherwise, it is intended that the compound includes both E and Z geometric isomers. Likewise all tautomeric forms are intended to be included.

U.S. Pat. No. 6,339,085 and U.S. Pat. No. 6,861,439 are herein incorporated by reference.

Generally, a P5P solution may be prepared by simply mixing P5P with a pharmaceutically acceptable solution, for example, buffered aqueous saline solution at an acidic or alkaline pH (because P5P is essentially insoluble in water, alcohol, and ether), at a temperature of at least room temperature and under sterile conditions. Preferably, a P5P solution is prepared immediately prior to administration to the mammal. However, if a P5P solution is prepared at a time more than immediately prior to the administration to the mammal, the prepared solution should be stored under sterile, refrigerated conditions. Furthermore, because P5P is light sensitive, a P5P solution should be stored in containers suitable for protecting a P5P solution from the light, such as amber-colored vials or bottles. Each amber colored vial or bottle can contain 250 mg of P5P or another compound described herein, or 750 mg of P5P or another compound described herein, or from about 200 to about 1000 mg of P5P or another compound described herein, for oral use. Alternatively, for parenteral use, each amber colored bottle or vial can contain about 5.00 mg of P5P or another compound described herein, about 23.33 mg of P5P or another compound described herein, or from about 2.00 to about 40.00 mg of P5P or another compound described herein.

According to an embodiment of invention, P5P, pyridoxine, pyridoxal, and pyridoxamine appropriately administered can have previously unexpected, highly beneficial effects in treating hypertrophy, hypertension congestive heart failure, myocardial ischemia, ischemia, heart disease, organ ischemia such as kidney ischemia, tissue ischemia, acute coronary syndrome, unstable angina, cognitive decline, or ischemia reperfusion injuries in mammals and in treatment of heart dysfunction subsequent to coronary occlusion, at lower concentrations of treatment than what was previously taught. According to embodiments of the invention, lower concentrations of P5P may have paradoxically higher effectiveness, when compared to higher concentrations of P5P previously taught. For example, P5P administered orally at a concentration of 250 mg/day was found to have higher effectiveness at reducing post-CABG morbidity and mortality than concentrations of 750 mg/day when a CK-MB level of greater than the pre-defined level of 50 ng/ml or a post-hoc analysis of 70 ng/ml is used as an indicator of myocardial ischemia. Concentrations of 250 mg/day (orally administered) and of 750 mg/day (orally administered) were both found to be effective at reducing post-CABG morbidity and mortality when a CK-MB level or greater than a post-hoc analysis of 100 ng/ml was used as an indicator of myocardial ischemia.

For illustrative purposes, a beneficial effect of administering P5P is demonstrated in the specific examples detailed below. Although the examples below discuss “high risk” patients, the results can be extended to all patients, regardless of risk, and, as such, the invention is useful for all patients, regardless of risk.

### EXAMPLES

### Example 1

### Oral Treatment in Post-CABG Patients

A randomized, double-blind placebo-controlled, dose-ranging, parallel-arrn multi-center study was undertaken, on high-risk patients undergoing CABG surgery with cardiopulmonary bypass.

Patients were identified as “high risk” if they had two or more of the following risk factors:
Age greater than 65 years;
Current smoker;

History of diabetes mellitus requiring treatment other than diet;
Evidence of left ventricular dysfunction or congestive heart failure;
History of a previous non-disabling stroke, transient ischemic attack, or carotid endarterectomy;
Urgent CABG intervention defined as the need to stay in the hospital (although the patient may be operated on within a normal scheduling routine);
History of myocardial infarction that occurred more than 48 hours but less than 6 weeks prior to a CABG surgery;
Prior peripheral artery surgery or angioplasty;
Moderate renal dysfunction defined as creatinine>133 μmol/L (1.5 mg/dl), but <250 μmol/L (2.8 mg/dl); and
Presence of at least one asymptomatic carotid artery stenosis (>50%) either in one or two carotid arteries.
   Approximately 900 high risk pre-CABG patients in 42 different treatment centers in North America were screened and randomized to three groups of approximately 300 patients each, prior to their bypass surgery, as follows. Patients were either placed in a control group (placebo), treated orally with 250 mg/day of P5P (250 mg/day), or treated orally with 750 mg/day of P5P (750 mg/day).

The first dose of study medication was administered at 3-10 hours prior to CABG surgery. In the event of surgery delay or rescheduling, a second pre-operative dose of P5P was administered so that all patients received study medication 3-10 hours before surgery. Treatment continued for 30 days after surgery (post operative day (POD) 30). Patients received follow-up evaluations up to and including postoperative day (POD) 4, on POD 30 and on POD 90.

Patients were measured for combined incidence of cardiovascular death, nonfatal myocardial infarction (MI), and nonfatal cerebral infarction up to and including post-operative day 30 (POD 30). Patients were also measured for nonfatal myocardial infarction alone. All deaths without an identifiable non-cardiovascular cause were considered of cardiovascular origin.

Cerebral infarction (stroke) was defined clinically as any new sudden onset focal neurological deficit lasting at least 24 hours as assessed by a neurologist and after neuroimaging (computed tomography [CT] brain scan or magnetic resonance imaging [MRI]) had excluded an intracerebral hemorrhage. All patients suspected of having a stroke or transient ischemic attack (TIA) received a neurological examination (conducted by a neurologist or internist with expertise in cerebral vascular disease) within 24 hours of onset of symptoms. All patients suspected of having a stroke were submitted to cerebral imaging.

When determining whether a patient had myocardial infarction, the following definitions were used:
A peak creatine kinase—myocardial band (CK-MB) above a certain threshold. Since different experts and different prior art clinical trials used different cut-offs for this threshold, three different thresholds were used to determine whether a patient had myocardial infarction—a post-hoc cut off threshold of 100 ng/ml (a peak CK-MB of 100 ng/ml or greater on days up to and including POD 4), a post-hoc cut off threshold of 70 ng/ml, and a predefined cut off threshold of 50 ng/ml was used;
A new q-wave evidence of myocardial infarction along with CK-MB of 35 ng/ml or above on days up to and including POD 4;
A peak CK-MB of 5× ULN (25 ng/ml) or above occurring after POD 4;
A new q-wave evidence of myocardial infarction that was not present at POD 4; or
A q-wave or non-q-wave myocardial infarction as identified by the investigator and confirmed by the Clinical Endpoint Committee.
   A. Myocardial Infarction Rates
   CK-MB Cut-off of 100 ng/ml

Oral treatment of both 250 mg/day and 750 mg/day were highly effective in reducing instances of myocardial infarction. See Table 1.

TABLE 1
Frequencies of myocardial infarction at POD 30

| | Placebo | 250 mg/day P5P | 750 mg/day P5P |
|---|---|---|---|
| Myocardial | 43 (14.4%) | 23 (7.6%) | 23 (7.6%) |
| Infarction | | | |
| No myocardial | 256 (85.6%) | 278 (92.4%) | 278 (92.4%) |
| infarction | | | |

Using CK-MB Cut-off of 70 ng/ml

Oral treatment of 250 mg/day was highly effective in reducing instances of myocardial infarction. Oral treatment of 750 mg/day was effective in reducing instances of myocardial infarction, but was not as effective as treatment of 250 mg/day. See Table 2.

TABLE 2
Frequencies of myocardial infarction at POD 30

| | Placebo | 250 mg/day P5P | 750 mg/day P5P |
|---|---|---|---|
| Myocardial | 54 (18.1%) | 34 (11.3%) | 38 (12.6%) |
| Infarction | | | |
| No myocardial | 245 (81.9%) | 267 (88.7%) | 263 (87.4%) |
| infarction | | | |

Using CK-MB Cut-off of 50 ng/ml

Oral treatment of 250 mg/day was highly effective in reducing instances of myocardial infarction. Oral treatment of 750 mg/day was not effective in reducing instances of myocardial infarction. See Table 3.

TABLE 3
Frequencies of myocardial infarction at POD 30

| | Placebo | 250 mg/day P5P | 750 mg/day P5P |
|---|---|---|---|
| Myocardial | 69 (23.1%) | 59 (19.6%) | 71 (23.6%) |
| Infarction | | | |
| No myocardial | 230 (76.9%) | 242 (80.4%) | 230 (76.4%) |
| infarction | | | |

B. Combined Incident/Composite Endpoint Rates

Frequencies of combined incidences (including myocardial infarction, cerebral infarction, or death), by treatment group, were as follows:

CK-MB Cut-off of 100 ng/ml

Oral treatment of either 250 mg/day or 750 mg/day was highly effective in reducing total incidents. Oral treatment of 250 mg/day was better at reducing total incidents as compared to treatment of 750 mg/day. See Table 4.

TABLE 4
Frequencies of myocardial infarction, cerebral infarction, or death, at
POD 30

| | Placebo | 250 mg/day P5P | 750 mg/day P5P |
|---|---|---|---|
| Myocardial | 49 (16.4%) | 31 (10.3%) | 34 (11.3%) |
| infarction, | | | |
| cerebral infarction | | | |
| or death | | | |
| No myocardial | 250 (83.6%) | 270 (89.7%) | 267 (88.7%) |
| infarction, cerebral | | | |
| infarction or death | | | |

CK-MB Cut-off of 70 ng/ml

Oral treatment of either 250 mg/day or 750 mg/day was highly effective in reducing total incidents. Oral treatment of 250 mg/day was better at reducing total incidents than treatment of 750 mg/day. See Table 5.

TABLE 5
Frequencies of myocardial infarction, cerebral infarction, or death, at
POD 30

| | Placebo | 250 mg/day P5P | 750 mg/day P5P |
|---|---|---|---|
| Myocardial | 60 (20.1%) | 41 (13.6%) | 47 (15.6%) |
| infarction, | | | |
| cerebral infarction | | | |
| or death | | | |
| No myocardial | 239 (79.9%) | 260 (86.4%) | 254 (84.4%) |
| infarction, cerebral | | | |
| infarction or death | | | |

CK-MB Cut-off of 50 ng/ml

Oral treatment of 250 mg/day was highly effective in reducing total incidents. Oral treatment of 750 mg/day did not reduce total incidents. See Table 6.

TABLE 6
Frequencies of myocardial infarction, cerebral infarction, or death, at
POD 30

| | Placebo | 250 mg/day P5P | 750 mg/day P5P |
|---|---|---|---|
| Myocardial | 75 (25.1%) | 65 (21.6%) | 77 (25.6%) |
| infarction, | | | |
| cerebral infarction | | | |
| or death | | | |
| No myocardial | 224 (74.9%) | 236 (78.4%) | 224 (74.4%) |
| infarction, cerebral | | | |
| infarction or death | | | |

### Example 2

### Parenteral Treatment in Post-CABG Patients

The randomized, double-blind placebo-controlled, dose-ranging, parallel-arm multi-center study on high-risk patients undergoing CABG surgery with cardiopulmonary bypass of Experiment 1 is repeated using a protocol as described in Experiment 1, with the difference that the placebo/drug is administered intravenously, in an intravenous dose pharmacokinetically approximating the dosage given enterally in Experiment 1.

Approximately 3000 high-risk pre-CABG patients are screened and randomized to 2 groups of approximately 1500 patients each, prior to their bypass surgery, as follows. Patients are either placed in a control group (placebo), treated intravenously with 5.00 mg/day of P5P, or treated intravenously with 23.33 mg/day of P5P.

Patients are identified as “high risk” if they had two or more of the following risk factors:

Age greater than 65 years;
Current smoker;
History of diabetes mellitus requiring treatment other than diet;
Evidence of left ventricular dysfunction or congestive heart failure;
History of a previous non-disabling stroke, transient ischemic attack, or carotid endarterectomy;
Urgent CABG intervention defined as the need to stay in the hospital (although the patient may be operated on within a normal scheduling routine);
History of myocardial infarction that occurred more than 48 hours but less than 6 weeks prior to CABG surgery;
Prior peripheral artery surgery or angioplasty;
Moderate renal dysfunction defined as creatinine>133 μmol/L (1.5 mg/dl), but <250 μmol/L (2.8 mg/dl); and
Presence of at least one asymptomatic carotid artery stenosis (>50%) either in one or two carotid arteries.

The first dose of study medication is administered at 3-10 hours prior to CABG surgery. In the event of surgery delay or rescheduling, a second pre-operative dose of P5P is administered so that all patients receive study medication 3-10 hours before surgery. Treatment continues for 30 days after surgery (post operative day (POD) 30). Patients receive follow-up evaluations up to and including postoperative day on POD 30 and on POD 90.

Patients are measured for combined incidence of cardiovascular death and nonfatal myocardial infarction (MI) up to and including post-operative day 30 (POD 30). Patients are also measured for nonfatal myocardial infarction alone.

All deaths without an identifiable non-cardiovascular cause are considered of cardiovascular origin.

Cerebral infarction (stroke) is defined clinically as any new sudden onset focal neurological deficit lasting at least 24 hours as assessed by a neurologist and after neuroimaging (computed tomography [CT] brain scan or magnetic resonance imaging [MRI]) excludes an intracerebral hemorrhage. All patients suspected of having a stroke or transient ischemic attack (TIA) receive a neurological examination (conducted by a neurologist or internist with expertise in cerebral vascular disease) within 24 hours of onset of symptoms. All patients suspected of having a stroke are submitted to cerebral imaging.

When determining whether a patient has myocardial infarction, the following definitions are used:
a peak creatine kinase—myocardial band (CK-MB) above a certain threshold. Since different experts and different prior art clinical trials used different cut-offs for this threshold, three different thresholds were used to determine whether a patient had myocardial infarction—a cut off threshold of 100 ng/ml (a peak CK-MB of 100 ng/ml or greater on days up to and including POD 4),
a new q-wave evidence of myocardial infarction along with CK-MB of 35 ng/ml or above on days up to and including POD 4;
a peak CK-MB of 5× ULN (25 ng/ml) or above occurring after POD 4;
a new q-wave evidence of myocardial infarction that was not present at POD 4; or
a q-wave or non-q-wave myocardial infarction as identified by the investigator and confirmed by the Clinical Endpoint Committee.
   A. Myocardial Infarction Rates
   CK-MB Cut-off of 100 ng/ml

Intravenous treatment of both 5.00 mg/day and 23.33 mg/day is highly effective in reducing instances of myocardial infarction.

B. Combined Incident/Composite Endpoint Rates

Frequencies of combined incidences (including myocardial infarction, cerebral infarction, or death), by treatment group, are as follows:

CK-MB Cut-off of 100 ng/ml

Intravenous treatment of either 5.00 mg/day or 23.33 mg/day is highly effective in reducing total incidents. Intravenous treatment of 5.00 mg/day is better at reducing total incidents as compared to treatment of 23.33 mg/day.

### Example 3

### Ability of P5P to Protect Against Cognitive Decline Following Cardiac Surgery

The ability of P5P to protect against cognitive decline following CABG surgery was evaluated through the administration of a battery of validated psychometric tests pre-operatively, 4 days after surgery, 30 days after surgery, and 90 days after surgery.

A randomized, double-blind placebo-controlled, dose-ranging, parallel-arm multi-center study was undertaken, on high-risk patients undergoing CABG surgery with cardiopulmonary bypass.

Patients were identified as “high risk” if they had two or more of the following risk factors:
Age greater than 65 years;
Diabetes mellitus;
History of congestive heart failure;
History of a previous non-disabling stroke, transient ischemic attack, or carotid endarterectomy;
Prior CABG surgery;
Urgent CABG intervention defined as the need to stay in the hospital (although the patient may be operated on within a normal scheduling routine);
History of myocardial infarction that occurred more than 48 hours but less than 6 weeks prior to CABG surgery; and
Presence of at least one asymptomatic carotid artery stenosis (>50%) either in one or two carotid arteries.

Approximately 900 high risk pre-CABG patients in 42 different treatment centers in North America were screened and randomized to three groups of approximately 300 patients each, prior to their bypass surgery, as follows. Patients were either placed in a control group (placebo), treated with 250 mg/day of P5P, (250 mg/day), or treated with 750 mg/day of P5P (750 mg/day). The first dose of study medication was administered at 3-10 hours prior to CABG surgery. In the event of surgery delay or rescheduling, a second pre-operative dose of P5P was administered so that all patients received study medication 3-10 hours before surgery. Treatment continued for 30 days after surgery (post operative day (POD) 30). Patients received follow-up evaluations up to and including postoperative day (POD) 4, on POD 30 and on POD 90.

Patients were measured to assess change in cognitive function by the effects of P5P on neurological events following CABG surgery by methods of Mini Mental State Exam and psychometric testing (grooved pegboard).

MMSE is a well validated test to screen cognitive function (Folstein, J Psychiatr Res. 1975 Nov.;12(3):189-98). It measures orientation, registration (immediate memory), short-term (but not long-term) memory, and language functioning. The test is scored from 0-30, with scores of 25-30 considered normal, scores of 18-24 indicative of mild cognitive impairment, and scores under 17 indicative of severe impairment (Crum, JAMA. 1993 May 12;269(18):2386-91). This test is appropriate to indicate the presence of cognitive impairment.

The MMSE was administered to all 900 patients at screening, post-operative day (POD) 30, and POD 90. The MMSE score at POD 90 was analyzed using an analysis of covariance (ANOCO) model, adjusting for the MMSE score at screening. The group x MMSE at screening interaction was found to be significant overall, with P=0.0034. The results with P5P treatment (either 250 or 750 mg/day) were tabulated in Table 7, according to screening MMSE score. Those patients with lower MMSE scores at screening (in particular, scores of 27 or lower) showed the greatest improvements, while those with higher scores at screening maintained their level of cognitive function. Improvements in MMSE score were more significant for patients in the 250 mg/day MC-1 dose group than for those in the 750 mg/day dose group, in particular in the patients with lower MMSE scores at screening.

TABLE 7
MMSE score results for MEND-CABG

| | | | P5P | P5P | |
|---|---|---|---|---|---|
| | | | 250 | 750 | |
| Randomization | | Placebo | mg/day | mg/day | p-value |
| group | | N = 299 | N = 301 | N = 302 | (ANOCO) |
| MMSE | N | 297 | 300 | 302 | |
| at | Mean ± SD | 28.69 ± | 28.59 ± | 28.75 ± | |
| Screen- | | 1.41 | 1.53 | 1.38 | |
| ing | | | | | |
| MMSE | N | 266 | 263 | 262 | |
| at | Mean ± | 28.58 ± | 28.57 ± | 28.79 ± | |
| POD 30 | SD | 2.98 | 2.35 | 2.05 | |
| MMSE | N | 255 | 252 | 250 | |
| at | Mean ± SD | 28.89 ± | 28.97 ± | 28.99 ± | |
| POD 90 | | 1.83 | 1.56 | 2.01 | |
| | Adjusted | | | | |
| | Mean ± SD | | | | |
| | MMSE | 26.62 ± | 27.74 ± | 27.41 ± | 0.0067 (placebo vs. |
| | screening = | 0.30 | 0.28 | 0.31 | 250 mg/day) |
| | 25 | | | | 0.0664 (placebo vs. |
| | | | | | 750 mg/day) |
| | MMSE | 27.22 ± | 28.08 ± | 27.83 ± | 0.0074 (placebo vs. |
| | screening = | 0.23 | 0.22 | 0.24 | 250 mg/day) |
| | 26 | | | | 0.0701 (placebo vs. |
| | | | | | 750 mg/day) |
| | MMSE | 27.83 ± | 28.42 ± | 28.25 ± | 0.0116 (placebo vs. |
| | screening = | 0.17 | 0.16 | 0.17 | 250 mg/day) |
| | 27 | | | | 0.0875 (placebo vs. |
| | | | | | 750 mg/day) |
| | MMSE | 28.44 ± | 28.76 ± | 28.67 ± | 0.0579 (placebo vs. |
| | screening = | 0.12 | 0.12 | 0.12 | 250 mg/day) |
| | 28 | | | | 0.1926 (placebo vs. |
| | | | | | 750 mg/day) |
| | MMSE | 29.05 ± | 29.10 ± | 29.08 ± | 0.7654 (placebo vs. |
| | screening = | 0.11 | 0.11 | 0.11 | 250 mg/day) |
| | 29 | | | | 0.8350 (placebo vs. |
| | | | | | 750 mg/day) |
| | MMSE | 29.66 ± | 29.44 ± | 29.50 ± | 0.2726 (placebo vs. |
| | screening = | 0.14 | 0.14 | 0.14 | 250 mg/day) |
| | 30 | | | | 0.4317 (placebo vs. |
| | | | | | 750 mg/day) |

These results show that in the MEND-CABG trial, treatment with P5P provided protection against the decline in overall neurocognitive function that is often associated with CABG surgery.

1. A method of treating hypertrophy, hypertension, myocardial ischemia, ischemic heart disease, myocardial infarction, congestive heart failure, organ ischemia, tissue ischemia, acute coronary syndrome, unstable angina, ischemia reperfusion injury, cerebral infarction, contractile dysfunction subsequent to myocardial infarction, or arrhythmia in a mammal comprising: administering a phannaceutical composition comprising a therapeutic amount of about 200 mg per day to about 300 mg per day of pyridoxal-5′-phosphate, wherein said administering is via oral administration.

2. The method of claim 1, wherein said pharmaccutical composition is administered prior to or during a procedure selected from the group consisting of bypass surgery, thrombolysis, endarterectomy, CABG, and angioplasty.

3. The method of claim 1, wherein said pharmaceutical composition is administered following a procedure selected from the group consisting of bypass surgery, thrombolysis, endarterectomy, CABG, and angioplasty.

4-5. (canceled)

6. The method of claim 1, wherein said therapeutic amount is about 250 mg per day.

7. (canceled)

8. A method of treating hypertrophy, myocardial ischemia, ischemic heart disease, myocardial infarction, congestive heart failure, organ ischemia, tissue ischemia, acute coronary syndrome, unstable angina, ischemia reperfusion injury, cerebral infarction, contractile dysfunction subsequent to myocardial infarction, or arrhythmia in a mammal comprising: administering a pharmaceutical composition comprising a therapeutic amount of about 3.00 mg per day to about 6.00 mg per day of pyridoxal-5′-phosphate, wherein said administering is via parenteral administration.

9. The method of claim 8, wherein said pharmaceutical composition is administered prior to or during a procedure selected from the group consisting of bypass surgery, thrombolysis, endarterectomy, CABG, and angioplasty.

10. The method of claim 8, wherein said pharmaceutical composition is administered following a procedure selected from the group consisting of bypass surgery, thrombolysis, endarterectomy, CABG, and angioplasty.

11-12. (canceled)

13. The method of claim 8, wherein said therapeutic amount is about 5.00 mg per day.

14. (canceled)

15. The method of claim 8, wherein the parenteral administration is an intravenous administration.

16. The method of claim 15, wherein the intravenous administration is a bolus injection.

17. The method of claim 15, wherein the intravenous administration is a continuous injection.

18. The method of claim 1 or 8, further comprising administering one or more additional compounds comprising at least one of a calcium channel blocker, a vasodilator, a diuretic, an alpha-blocker, or a beta-blocker, wherein said method treats hypertrophy.

19. The method of claim 1 or 8, further comprising administering one or more additional compounds comprising at least one of a HMG-CoA Reductase inhibitor, a vasodilating agent, a diuretic, an ACE inhibitor, a beta-blocker, an angiotensin II receptor antagonist, a calcium channel blocker, an anticoagulant, an adenosine diphosphate receptor antagonist, a glycoprotein IIb/IIIa receptor antagonist, or an alpha blocker, wherein said method treats myocardial ischemia.

20. The method of claim 1 or 8, further comprising administering one or more additional compounds comprising at least one of a cardiotonic agent, a cardiac glycoside, a vasodilating agent, a nitrate, a nitrite, an ACE inhibitor, an angiotensin II receptor antagonist, a mineralocorticoid, a diuretic, an alpha-adrenergic receptor antagonist, a mineralocorticoid, or a diuretic, wherein said method treats congestive heart failure.

21. The method of claim 1 or 8, further comprising administering one or more additional compounds comprising at least one of a GB IIb/IIIa receptor antagonists, an anti-platelet agent, a calcium channel blocker, a beta blocker, a nitrate, or an anticoagulant, wherein said method treats acute coronary syndrome.

22. The method of claim 1 or 8, further comprising administering one or more additional compounds comprising at least one of a HMG-CoA Reductase Inhibitor, a GP IIb/IIIa Receptor antagonist, an anticoagulant, an adenosine diphosphate receptor antagonist, or heparin, wherein said method treats unstable angina.

23. The method of claim 1 or 8, further comprising administering one or more additional compounds comprising at least one of a HMG-CoA Reductase inhibitor, a vasodilating agent, a diuretic, an ACE inhibitor, a beta-blocker, an angiotensin II receptor antagonist, a calcium channel blocker, an anticoagulant, an adenosine diphosphate receptor antagonist, a glycoprotein IIb/IIIa receptor antagonist, an alpha blocker, or an anticoagulant, wherein said method treats ischemia reperfusion injury.

24. The method of claim 1 or 8, further comprising administering one or more additional compounds comprising at least one of a HMG-CoA reductase inhibitor, an ACE inhibitor, an angiotensin II receptor antagonist, an adenosine diphosphate receptor antagonist, a glycoprotein IIb/IIIa receptor antagonist, an anticoagulant, agent, or a calcium channel blocking agent, wherein said method treats cerebral infarction.

25. The method of claim 1 or 8, further comprising administering one or more additional compounds comprising at least one of an antiarrhythmic, heparin, warfarin, or digoxin, wherein said method treats arrhythmia.

26. The method of claim 1 or 8, further comprising administering one or more additional compounds comprising at least one of a HMG-CoA reductase inhibitor, an ACE inhibitor, an anticoagulant, an adenosine diphosphate receptor antagonist, a glycoprotein IIb/IIIa receptor antagonist, an alpha-adrenergic receptor antagonist, a beta-blocker, an antiarrhythmic, a hypotensive agent, a direct vasodilator, or a calcium channel blocking agent, wherein said method treats myocardial infarction.

27. The method of claim 1 or 8, further comprising administering one or more additional compounds comprising at least one of an ACE inhibitor, an angiotensin II receptor antagonist, a renin inhibitor, an endothelin selective receptor antagonist, a diuretic, a alpha blocker, a calcium channel blocker, or a vasodilating agent, wherein said method treats hypertension.

28. The method of claim 1 or 8, further comprising administering one or more additional compounds comprising at least one of a HMG-CoA Reductase inhibitor, a vasodilating agent, a diuretic, an ACE inhibitor, a beta-blocker, an angiotensin II receptor antagonist, a calcium channel blocker, an anticoagulant, an adenosine diphosphate receptor antagonist, a glycoprotein IIb/IIIa receptor antagonist, or an alpha blocker, wherein said method treats ischemic heart disease.

29. A composition comprising about 250 mg of pyridoxal-5′-phosphate, and a pharmaceutically acceptable excipient.

30. The composition of claim 29 further comprising an additional therapeutic compound.

31-32. (canceled)

33. A composition comprising about 5.00 mg of pyridoxal-5′-phosphate, and a phannaceutically acceptable excipient.

34. The composition of claim 33 further comprising an additional therapeutic compound.

37. A kit comprising:
(a) a pharmaceutical preparation for oral administration comprising pyridoxal-5′-phosphate; and
(b) instructions for the administration of said preparation, said instructions specifying that said preparation should be administered about 200 mg to about 300 mg per day.

38. The kit of claim 37, wherein the instructions further specify that the preparation should be administered prior to or during a procedure selected from the group consisting of bypass surgery, thrombolysis, endarterectomy, CABG, and angioplasty.

39. The kit of claim 37, wherein the instructions further specify that the preparation should be administered after a procedure selected from the group consisting of bypass surgery, thrombolysis, endarterectomy, CABG, and angioplasty.

40. (canceled)

41. The kit of claims 37, wherein the instructions further specify that said preparation should be administered in a dosage of about 250 mg per day.

42-43. (canceled)

44. The kit of claim 37, further comprising a phannaceutical preparation for parenteral administration comprising pyridoxal-5′-phosphate, wherein the instructions further specify use of the parenteral phannaceutical preparation, followed by use of the oral pharmaceutical preparation.

45. A kit comprising:
(a) a pharmaceutical preparation for parenteral administration comprising pyridoxal-5′-phosphate;
(b) instructions for the administration of said preparation, said instructions specifying that said preparation should be administered about 3.00 mg to about 6.00 mg per day.

46. The kit of claim 45, wherein the instructions further specify that the preparation should be administered prior to or during a procedure selected from the group consisting of bypass surgery, thrombolysis, endarterectomy, CABG, and angioplasty.

47. The kit of claim 45, wherein the instructions further specify that the preparation should be administered after a procedure selected from the group consisting of bypass surgery, thrombolysis, endarterectomy, CABG, and angioplasty.

48. (canceled)

49. The kit of claim 45 wherein the instructions further specify that said preparation should be administered in a dosage of about 5.00 mg per day.

50-51. (canceled)
